(19) United States
(12) Patent Application Publication
(10) Pub. No.: US 2006/0137106 A1
(43) Pub. Date: Jun. 29, 2006

(54) Method and apparatus for dispensing solution on a cleaning cloth

(76) Inventor: Avery Goodman, Greendale, WI (US)

Correspondence Address: SEAN KAUFHOLD, P. O. BOX 89626, SIOUX FALLS, SD, 57109, US

(21) Appl. No.: 11/177,693

(22) Filed: Jul. 11, 2005

Related U.S. Application Data
(63) Continuation-in-part of application No. 10/705,528, filed on Nov. 12, 2003, now Pat. No. 7,004,659.

(51) Int. Cl. A01H 5/02; D06B 1/00

(52) U.S. Cl. 8/148; 68/200

(57) ABSTRACT

An apparatus for dispensing solution on a cleaning cloth includes a container that has a bottom wall, a top wall and a peripheral wall. An opening extends through the top wall. A pump assembly is configured for selectively pumping a fluid outwardly out of the housing's interior. A fluid receiving housing includes a lower wall, an upper wall and a perimeter wall. The upper wall has a plurality of holes extending therethrough. The pump assembly is attached to the lower wall and is fluidly coupled to the housing. The housing defines an actuator for pumping fluid out of the container. A sponge material mounted within the housing absorbs fluid pumped into the housing and expels the fluid through the holes when the pump assembly is actuated. A cleaning cloth positioned on an outer surface of the upper wall absorbs the fluid expelled through the holes.

This application is a continuation in part of application Ser. No. 10/705,528 filed on Nov. 12^{th}, 2003.

### BACKGROUND OF THE INVENTION

1. Field of the Invention

The present invention relates to solution dispensing devices and more particularly pertains to a new solution dispensing device for positioning fluid, and more particularly a soap solution, on a cleaning cloth.

2. Description of the Prior Art

The use of solution dispensing devices is known in the prior art. While these devices fulfill their respective, particular objectives and requirements, the need remains for a device that is particularly suited for providing a controlled amount of cleaning solution on a wide area of a section of a cleaning cloth so that the cleaning cloth is more effective for cleaning.

### SUMMARY OF THE INVENTION

The present invention meets the needs presented above by generally comprising a container that has a bottom wall, a top wall and a peripheral wall that is attached to and extends between the top and bottom walls. An opening extends through the top wall and into an interior of the container. A pump assembly is configured for selectively pumping a fluid outwardly out of the interior. A fluid receiving housing includes a lower wall, an upper wall and a perimeter wall extending between and is attached to the lower and upper walls. The upper wall has a plurality of holes extending therethrough. The pump assembly is attached to the lower wall and is fluidly coupled to the housing. The housing defines an actuator for pumping fluid out of the container when the housing is urged toward the container. A sponge material is removably mounted within the housing and abuts the upper wall. The sponge absorbs fluid pumped into the housing and expels the fluid through the holes when the pump assembly is actuated. A cleaning cloth may be positioned on an outer surface of the upper wall so that fluid expelled through the holes is positioned on the cleaning cloth.

There has thus been outlined, rather broadly, the more important features of the invention in order that the detailed description thereof that follows may be better understood, and in order that the present contribution to the art may be better appreciated. There are additional features of the invention that-will be described hereinafter and which will form the subject matter of the claims appended hereto.

The objects of the invention, along with the various features of novelty which characterize the invention, are pointed out with particularity in the claims annexed to and forming a part of this disclosure.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a perspective view of a method and apparatus for dispensing solution on a cleaning cloth according to the present invention.

FIG. 2 is a cross-sectional view taken along line 2-2 of FIG. 1 of the present invention.

FIG. 3 is a cross-sectional view taken along line 3-3 of FIG. 2 of the present invention.

FIG. 4 is a schematic cross-sectional view of the housing of the present invention.

### DESCRIPTION OF THE PREFERRED EMBODIMENT

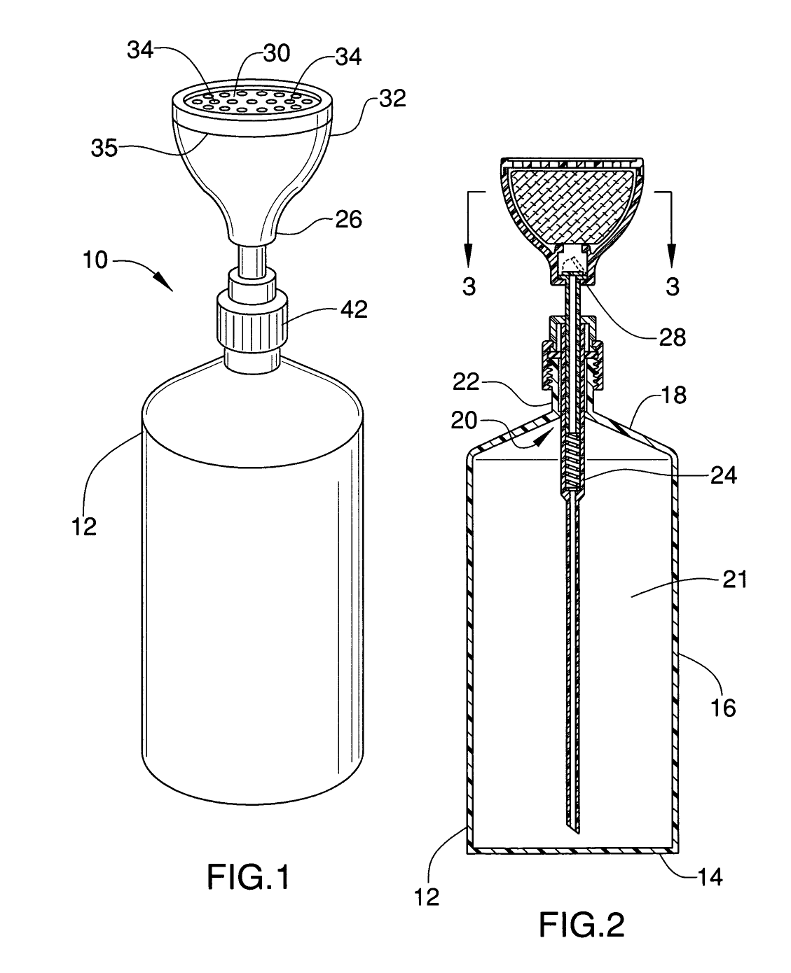

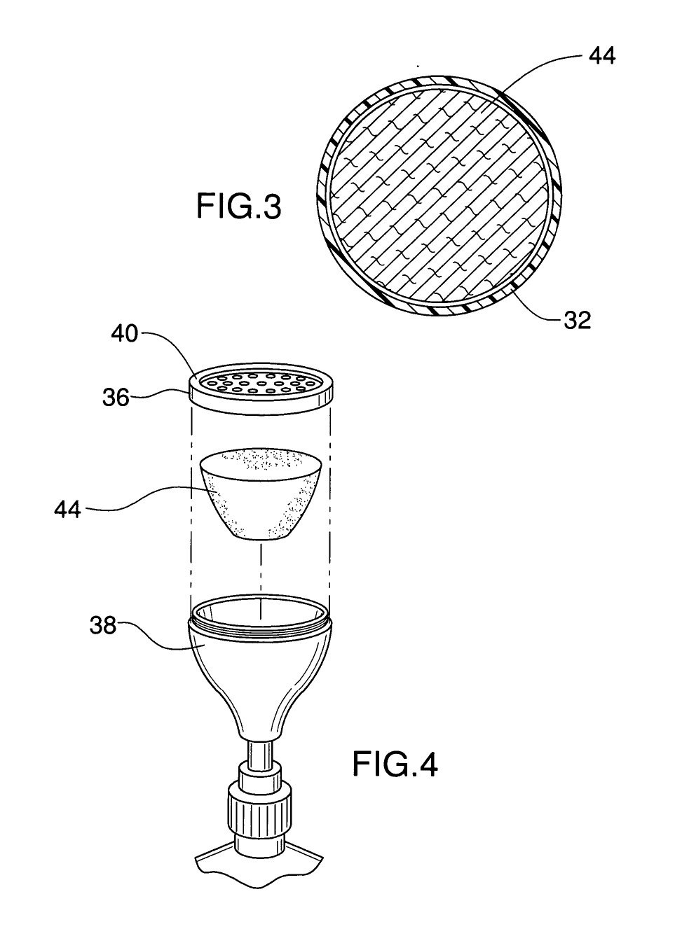

With reference now to the drawings, and in particular to FIGS. 1 through 4 thereof, a new solution dispensing device embodying the principles and concepts of the present invention and generally designated by the reference numeral 10 will be described.

As best illustrated in FIGS. 1 through 4 a container 12 has a bottom wall 14, a top wall 18 and a peripheral wall 16 that is attached to and extends between the top 14 and bottom 18 walls. An opening 20 extends through the top wall 14 and into an interior 21 of the container 12. A threaded neck 22 is attached to and extends upwardly from a peripheral edge of the opening 20.

A pump assembly 24 configured for selectively pumping a fluid outwardly out of the interior 21 of the container 12 is fluidly mounted to the container 12. The pump assembly 24 is generally conventional in nature.

A fluid receiving housing 26 includes a lower wall 28, an upper wall 30 and a perimeter wall 32 that extends between and is attached to the lower 28 and upper 30 walls. The upper wall 30 has a plurality of holes 34 extending therethrough. The pump assembly 24 is attached to the lower wall 28 and is fluidly coupled to the housing 26. The housing 26 defines an actuator for pumping fluid out of the container 12 when the housing 26 is urged toward the container 12. The upper wall 30 has a circular shape. The perimeter wall 32 has a break 35 therein and an upper portion 36 and a lower portion 38 of the housing 26 are defined. The upper portion 36 is threadably couplable to the lower portion 38. A peripheral lip 40 is attached to and extends along an edge of the upper wall 30. The lip 40 extends upwardly from the upper wall 30.

A coupler 42 is attached to the pump assembly 24 for releasably coupling the housing 26 to the neck 22 so that the pump assembly 24 extends into the interior 21 of the container 12.

A sponge material 44 is removably mounted within the housing 26 and abuts the upper wall 30. The sponge 44 absorbs fluid pumped into the housing 26 and expels the fluid through the holes 34 when the pump assembly 24 is actuated.

In use, the housing 26 is depressed to saturate the sponge 44 with fluid, which is preferably a cleaning solution. A cleaning cloth may be positioned on an outer surface of the upper wall 30 so that fluid expelled through the holes 34 is positioned on the cleaning cloth. The cleaning cloth may be comprised of a paper material such as paper towels or toilet paper, or may be comprised of natural and/or synthetic cloth fibers.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A cleaning solution application device for selectively positioning cleaning solution on a section of a cleaning cloth, said device comprising:
a container having a bottom wall, a top wall and a peripheral wall being attached to and extending between said top and bottom walls, an opening extending through said top wall and into an interior of said container;
a pump assembly configured for selectively pumping a fluid outwardly out of said interior;
a fluid receiving housing including a lower wall, an upper wall and a perimeter wall extending between and being attached to said lower and upper walls, said upper wall having a plurality of holes extending therethrough, said pump assembly being attached to said lower wall and being fluidly coupled to said housing, said housing defining an actuator for pumping fluid out of said container when said housing is urged toward said container;
a sponge material being removably mounted within said housing and abutting said upper wall, said sponge absorbing fluid pumped into said housing and expelling the fluid through said holes when said pump assembly is actuated; and
wherein a cleaning cloth may be positioned on an outer surface of said upper wall such that fluid expelled through said holes is positioned on the cleaning cloth.

2. The device according to claim 1, further including a threaded neck being attached to and extending upwardly from a peripheral edge of said opening, a coupler being attached to said pump assembly for releasably coupling said housing to said neck such that said pump assembly extends into the interior of said container.

3. The device according to claim 1, wherein said upper wall has a circular shape.

4. The device according to claim 1, wherein said perimeter wall has a break therein and an upper portion and a lower portion of said housing being defined, said upper portion being threadably coupled to said lower portion.

5. The device according to claim 4, further including a peripheral lip being attached to and extending along an edge of said upper wall, said lip extending upwardly from said upper wall.

6. The device according to claim 5, further including a peripheral lip being attached to and extending along an edge of said upper wall, said lip extending upwardly from said upper wall.

7. A cleaning solution application device for selectively positioning cleaning solution on a section of a cleaning cloth, said device comprising:
a container having a bottom wall, a top wall and a peripheral wall being attached to and extending between said top and bottom walls, an opening extending through said top wall and into an interior of said container, a threaded neck being attached to and extending upwardly from a peripheral edge of said opening;
a pump assembly configured for selectively pumping a fluid outwardly out of said interior;
a fluid receiving housing including a lower wall, an upper wall and a perimeter wall extending between and being attached to said lower and upper walls, said upper wall having a plurality of holes extending therethrough, said pump assembly being attached to said lower wall and being fluidly coupled to said housing, said housing defining an actuator for pumping fluid out of said container when said housing is urged toward said container, said upper wall having a circular shape, said perimeter wall having a break therein and an upper portion and a lower portion of said housing being defined, said upper portion being threadably coupled to said lower portion;
a peripheral lip being attached to and extending along an edge of said upper wall, said lip extending upwardly from said upper wall;
a coupler being attached to said pump assembly for releasably coupling said housing to said neck such that said pump assembly extends into the interior of said container;
a sponge material being removably mounted within said housing and abutting said upper wall, said sponge absorbing fluid pumped into said housing and expelling the fluid through said holes when said pump assembly is actuated; and
wherein a cleaning cloth may be positioned on an outer surface of said upper wall such that fluid expelled through said holes is positioned on the cleaning cloth.
